(19) United States
(12) Patent Application Publication
(10) Pub. No.: US 2016/0238568 A1
(43) Pub. Date: Aug. 18, 2016

(54) TYPING AND IMAGING OF BIOLOGICAL AND NON-BIOLOGICAL MATERIALS USING QUANTITATIVE ULTRASOUND

(71) Applicants: Ernest J. Feleppa, Rye, NY (US); Jonathan Mamou, New York, NY (US)

(72) Inventors: Ernest J. Feleppa, Rye, NY (US); Jonathan Mamou, New York, NY (US)

(73) Assignee: Riverside Research Institute, New York, NY (US)

(21) Appl. No.: 14/624,769

(22) Filed: Feb. 18, 2015

(51) Int. Cl. G01N 29/36; G01N 29/44; A61B 8/14; A61B 5/00; A61B 8/08

(52) U.S. Cl. CPC G01N 29/36; A61B 5/7267; A61B 8/08; A61B 8/5223; A61B 8/14; G01N 29/44; G01N 2291/044

(57) ABSTRACT

An ultrasonic material-evaluation or classification method using spectral and envelope-statistics variables from backscattered ultrasound echo signals combined with global variables. This classification method can be applied to any organ or tissue among biological materials and any non-biological material that produces backscattered signals as a result of microscopic internal inhomogeneities such as a crystalline structure.

### PRIORITY AND RELATED APPLICATION

U.S. Pat. No. 6,238,342 ('342 patent) is related to this application and is hereby incorporated by reference in its entirety.

### FIELD OF THE INVENTION

The present invention relates to improved typing and imaging of biological and non-biological materials by combining quantitative-ultrasound (QUS) estimates based on the statistics of the envelope of echo signals generated using pulse-echo ultrasound with QUS estimates based on the normalized power spectra of echo signals generated using pulse-echo ultrasound and global variables associated with the material of interest.

### BACKGROUND OF THE INVENTION

The improvements described herein generalize the definition of QUS by adding:

(1) estimates based on variables of the statistics of the envelope of linearly amplified, radio-frequency ultrasound echo signals backscattered from biological or non-biological materials to

(2) estimates based on variables of normalized power spectra of linearly amplified, radio-frequency ultrasound echo signals in previously described QUS methods such as those shown in the '342 patent, and optionally

(3) one or more global variables such as clinical data, e.g., antigen level or patient age, when typing tissue or such as material properties, e.g., acoustic attenuation or mass density, when typing non-biological material.

The improvements also generalize the application of the covered QUS methods to any and all materials in which pulse-echo ultrasound produces echo signals within the material and where such echo signals result from spatial variations in the acoustical impedance of the material on a scale of fractions to multiples of the incident acoustical-pulse wavelength.

Although specific clinical examples are cited herein to illustrate applications of the described method, the method is applicable to a very broad range of material-typing and imaging applications in addition to the cited clinical, tissue-typing and imaging applications. Examples of potential non-biological material-typing and imaging applications include, but are not limited to, assessment of composite quality, fiber density in fiber-reinforced plastics, crystalline-material properties, particle size and concentration in liquid suspensions, etc. An example of a potential non-clinical, biological-material typing and imaging includes, but is not limited to, beef-quality grading. Examples of potential clinical applications include, but are not limited to, distinguishing among healing, non-healing and infected wounds; distinguishing between ischemic and non-ischemic myocardium; distinguishing among progressing, static, and regressing lesions; distinguishing between lesions that are responsive to treatment and those that are unresponsive to treatment; etc. Furthermore, in clinical applications, the method may be able to grade conditions such as, for example, the degree of treatment response, severity of ischemia, extent of infection, rate of healing, depth of burns, pressure or friction-ulcer status, progression of disease; etc.

For example, two salient, representative clinical applications are detection and imaging of cancer in the prostate gland or of metastases in lymph nodes. Reliable detection of primary-cancer foci in the prostate or metastatic foci in lymph nodes is critical for staging the disease and planning its treatment. The described method of cancer detection analyzes raw ultrasound echo-signal data in two- or three-dimensions (2D or 3D) in combination with global clinical variables such as serum PSA (prostate-specific antigen) values in the case of prostate cancer or primary-tumor type in the case of lymph-node metastases to generate³D images that depict cancerous foci in the prostate or lymph nodes and thereby that reliably detect, characterize, and localize metastatic regions.

A reliable method using spectrum-analysis-based QUS to characterize and type biological tissue is described in the '342 patent and is incorporated herein by reference. The '342 patent describes a method that combines spectrum-analysis-based QUS variables (i.e., the slope, intercept, mid-band variables of the so-called normalized power spectrum and also the effective scatterer size and so-called acoustic concentration estimates that are derived from the spectral variables and known ultrasound-system properties) with global clinical data.

The improvements described herein additionally combine variable values of the envelope statistics of ultrasound echo signals derived from the tissue of interest with the spectrum-analysis-based variable values and global variables described in the '342 patent, and/or described herein.

### SUMMARY OF THE INVENTION

Quantitative ultrasound (QUS) is generalized to add estimates derived from the statistics of envelope detected echo-signal data, to estimates based on variables of the normalized power spectra of echo signals generated by pulse-echo ultrasound in 2D or 3D, combined with the values of global variables of the biological or non-biological material to characterize said biological or non-biological material based on its properties at microscopic levels on a scale that ranges from a fraction to a small multiple of the wavelength of the incident ultrasound pulse. Variables of the normalized power spectrum are computed from acquired, linearly amplified, radio-frequency echo signals backscattered from the material being evaluated. Variables also are derived by computing variables of the statistics of the envelope of the backscattered signals. The statistics of the envelope of the echo signal are modeled using distributions such as, but not limited to, Nakagami and homodyned-K distributions. The combined set of spectral and statistics-based variables comprise the QUS components of the described improvements to the '342 patent. Once variable computation is complete, estimate values can be combined further with one or more global variables associated with the material being evaluated for the purpose of classifying, grading, or otherwise characterizing the material using linear or non-linear classification methods such as, but not limited to, linear-discriminant methods, artificial neural networks, nearest-neighbor algorithms, and support-vector machines. Subsequently, color-coded QUS-based images can be constructed from QUS values or from classification values derived from them on a pixel-by-pixel basis to produce 2D depictions or on a voxel-by-voxel basis to produce 3D depictions to visualize, for example, an entire lymph node or a volume of a fiber-reinforced plastic.

The invention employs in part an ultrasound apparatus for performing material classification as disclosed in the '342 patent and that is incorporated herein by reference. Such an apparatus includes a so-called pulse-echo ultrasound scanner for acquiring original, so-called radio-frequency or “RF” echo signals backscattered by the material being tested, such as biological tissue or non-biological material such as fiber-reinforced plastic. Analog RF echo signals are presented to a digitizer that converts the RF signals to digital signals, representing a plurality of spatial points in a scanned plane. A digital processor operatively computes so-called normalized power spectra from the digital signals provided by the digitizer and extracts spectral variables that characterize the original RF signals. Concurrently, the processor computes the envelope of the RF signals and, from the computed envelope, it computes the associated statistics of the envelope. An input device is also included for providing the values of global variables to the processor. Alternatively, global variable values may be computed directly from the ultrasound data, e.g., specimen shape factors, specimen volume, etc. A classifier that is responsive to at least a portion of the set of spectral variables, at least a portion of the set of envelope statistics, and at least a portion of the set of global variables, is employed to assign a material-classification score to the spatial points of the plane or volume covered by the ultrasound scan. A display may be provided for displaying the assigned classification scores in a color- or gray-scale encoded manner in a 2D or 3D image, but a display is not required to practice the invention described herein.

### BRIEF DESCRIPTION OF THE DRAWINGS

Further objects, features and advantages of the invention will become apparent from the following detailed description taken in conjunction with the accompanying figures showing illustrative embodiments of the invention, in which

FIG. 1 is a block diagram of an ultrasound imaging system formed in accordance with the present invention;

FIG. 2 is a flow chart depicting the process of training a classifier and optionally generating a derivative thereof, such as a look-up table, for performing tissue classification in accordance with the present invention; and

FIG. 3 is a flow chart depicting the process of generating images using computed spectral estimates, statistical estimates, global-variable values, and a classifier or a derivative thereof, such as a look-up table, to distinguish the material being evaluated into a number of classifications, such as most-likely tissue type or levels of suspicion of cancer.

### DETAILED DESCRIPTION OF THE INVENTION

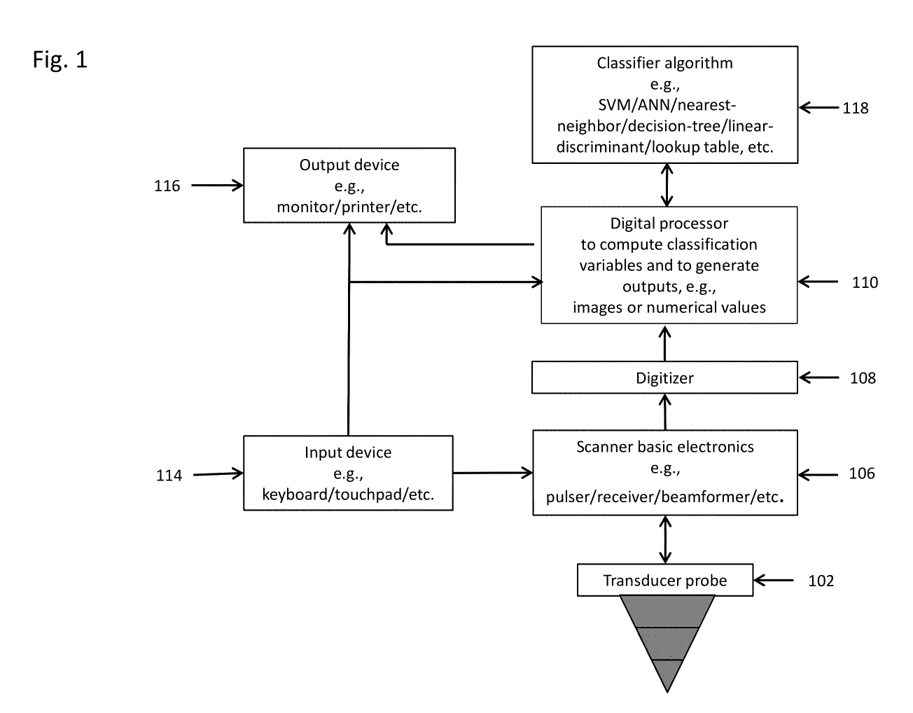

FIG. 1 is a general block diagram of system hardware that could be used in conjunction with the present invention. The general system hardware includes an ultrasonic transducer probe 102 operatively coupled to an ultrasound scanner 106. The signals from the scanner 106 are converted from an analog RF signal to a digital signal by a digitizer 108 operating under the control of a processor 110. In an exemplary embodiment, the digitizer 108 operates at a 50 MHz sampling rate to acquire, for example, 2500, 8-bit samples of echo-signal data in a frequency band extending from 3.5 to 8.0 MHz along each of 318 scan lines in a 112 degree scanning sector. A small set of these sample points represents a pixel in an ultrasound image. This results in a sector with a radius of about 3.6 cm. The digital samples are stored in a computer memory under the control of processor 110.

The processor 110 may be included in a desktop computer or workstation interfaced with the scanner 106 or may be integral to the scanner 106. A high-speed processor is required for real-time imaging. In a laboratory setting, the digitizer 108 can take the form of digital sampling oscilloscope. However, for commercially produced clinical or material-testing scanners, the digitizer 108 will generally be integrated into the scanner 106 or will be integrated with a subsystem along with processor 110. Optionally, the digitizer 108 and associated interface circuitry to a conventional ultrasound scanner 106 can be provided on a computer interface card for a conventional desktop computer system or workstation in which the processor 110 resides.

The system of FIG. 1 also includes an input device 114 for manually entering data into the system 100. The input device may take the form of a keyboard, touch screen, or digital pointing device, such as a computer mouse, used in conjunction with a display device 116. The display device 116 can be a standard computer monitor or printer.

A critically important element of the present invention is a classifier 118. The classifier 118, which may be implemented as a look-up table, trained neural network, nearest neighbor model, support-vector machine, and the like, is developed or trained using conclusive material-property data variables (e.g., histological data from biopsy results or known reinforced-plastic material properties) with matching RF-signal spectral-variable values, envelope-signal statistics-variable values, and global-variable values 110.

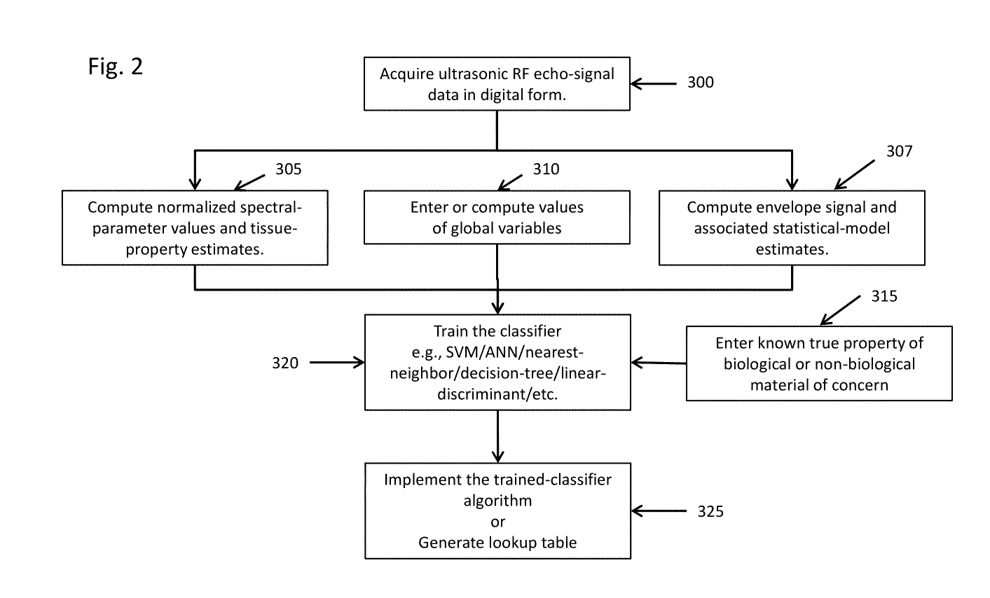

FIG. 2 is a flow chart illustrating exemplary steps used to develop and train the classifier 118. Preferably, the classifier 118 takes the form of a “trained” non-linear classifier, such as a support-vector machine, but other linear and non-linear classifiers also can be employed. In order to develop a “trained” non-linear classifier 118, training data are required. These training data must include a sufficiently large number of independent records to provide adequate statistical stability; each record includes spectral- and statistical-variable values computed from the RF echo-signal data (steps 300, 305 and 307), global-variable values, for instance known reinforced-plastic material properties (such as mass density) or clinical data (such as patients' age, ethnicity, prior medical history, or, in the case of prostate-cancer detection, PSA level) (step 310) and corresponding “gold-standard” data (step 315), for instance, the clinical “gold standard” histologically established tissue properties (such as biopsy results) for each record. The set of spectral-variable values and envelope-statistics-variable values are matched with gold-standard data (such as histological determinations of the actual tissue in the biopsy sample or independently measured actual fiber content in a reinforced-plastic).

The classifier is trained in step 320 and the trained-classifier algorithm is implemented in step 325. Various algorithms for classifiers are known and will not be further described herein.

After the RF backscatter data are acquired (step 300), digital signal analysis is performed on the acquired data to compute the set of QUS estimates consisting of spectral and envelope-statistics variable values representing the data (steps 305 and 307). In the '342 patent, spectral variables that have been found to be of interest in cancer diagnostics include the slope, intercept and mid-band values of a linear regression approximation to the normalized power spectrum of the backscattered radiofrequency (RF) echo signals. Scatter-property estimates, such as the effective scatterer size and so-called acoustic concentration derived from the spectral variables and known ultrasound-system properties, also have proven to be of value in classifying tissue. These estimates are computed from the echo signals in a user-specified analysis region of interest (ROI). Four additional variables have been shown to improve classification; these are derived by fitting envelope-amplitude distribution models to the envelope statistics of the backscattered envelope signals statistics based on the Nakagami and homodyned-K statistical models (step 307). The four new QUS variables associated with these two models are a, Q, k and g. However, other statistical models and associated variables can be used. The method is not limited to these statistical models.

To compute the variables for training the classifier, a user-specified ROI is applied to the acquired RF data to select a set of samples of the RF signals that spatially corresponds to the region from which the gold-standard determination is made, for instance the tissue region exactly matching the tissue sampled by the biopsy procedure or the portion of the reinforced plastic that will be exposed for fiber-density determination. The calculation of the spectral variables from RF echo-signal data defined in the '342 patent has been widely publicized and is a technique that is well known in the art of ultrasound diagnostics. The statistics of envelope-detected echo signals are quantified using Nakagami and homodyned-K or other applicable statistical models. For instance, envelope-statistics variables, a, and Q, are obtained by fitting a Nakagami probability density function to that of the envelope of RF data within the ROI, while the variables, k and μ, are obtained by fitting the homodyned-K probability density function to that of the envelope of RF data within the ROI.

Tissue classification using spectral and derived variables, envelope statistics from ultrasound echo signals combined with global-variable values is an approach that can be generalized for application to any organ or tissue and also to a wide variety of non-biological materials. In clinical applications, the approach can be used to monitor therapy or disease progression, and can be applied to diffuse disease, healing processes, etc., and is not limited to cancer applications. Examples of analysis using the present invention in clinical applications are provided below.

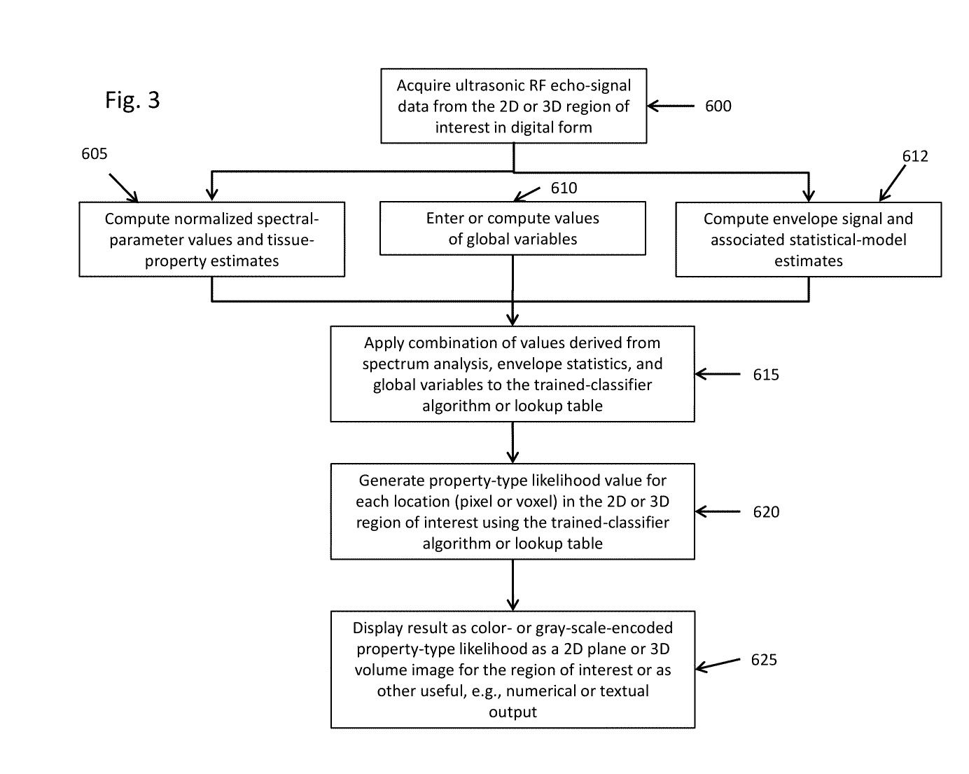

FIG. 3 is a flow chart illustrating an exemplary clinical application of the present invention. Global variables such as clinical data or non-biological data are input to the system (step 610) by a person using the input device 114. RF echo-signal data are acquired and digitized (step 600). Spectral-variables and scatterer-property values along with variables of the envelope statistics comprising the full set of QUS-variable values are extracted and computed (steps 605 and 612). The selected global-variable and QUS-variable values are applied as input variables to the classifier 118, then normalized to the input range of the classifier 118 on a pixel by pixel basis, such that each pixel of the sampled ultrasonic scan is assigned a classifier-score value (step 615). For real-time operation, the classifier 118 can take the form of a look-up table whose values are derived from a trained linear or non-linear classifier. Alternatively, if the processor 110 is sufficiently powerful, the classifier 118 may omit the look-up table and the classifier-score value can be assigned for each pixel by applying the input variable values to the optimal classifier and computing classifier-score values directly using the trained classifier.

In step 620, the property-type likelihood value for each location (pixel or voxel) in the 2D or 3D ROI is generated using the trained-classifier algorithm or look-up table. In step 625, a display is generated that can be color or gray scale-encoded, property-type likelihood result. The display can be a 2D plane or a 3D volume for the ROI, or as any other useful output.

Although evaluating the classifier scores over a broad range of values is important to account for a possibly large number of variables involved in the process, the display need not show each individual classifier-score value as a unique display variable. The range of classifier-score values can be grouped into a plurality of ranges that correspond to most-likely material categories, for example, most-likely tissue types, a number of levels of suspicion (LOS) for cancer in a clinical application or similar categories for non-biological material. Each of the LOS ranges is assigned a unique image characteristic, such as a color or grey scale value, for pixels within that range for displaying the results (step 625).

Although the present invention has been partially described in connection with lymph nodes, the present techniques are generally applicable to any region of a body where ultrasound backscattered echo signals can be obtained and also are applicable to non-clinical applications, e.g., those involving research with experimental animals, and to evaluations of non-biological materials. Each specific material type requires its own classifier 118 appropriately trained using a suitable database of global-variable values, QUS variable values (spectral and envelope statistical), and gold-standard results for the target application.

In clinical applications, in addition to classifying tissue in accordance with a level of suspicion or likelihood for cancer, various other tissue types or changes in tissue characteristics can be evaluated with the present invention. For example, changes in tissue in response to therapy, disease progression, injury severity, injury healing, and the like can be assessed. In practice, a clinical device will have a menu of applications to select from as part of the initial instrument set up.

In non-clinical biological applications, for example those involving experimental animals, various other tissue types or changes in tissue characteristics can be evaluated with the present invention. For example, as in clinical applications, changes in tissue in response to therapy, disease progression, injury severity, injury healing, and the like can be assessed quantitatively by assessing classifier-score values. In practice, a device for non-clinical, biological use will have a menu of applications to select from as part of the initial instrument set up.

In non-biological, materials-evaluation applications, in addition to classifying material properties, changes in material characteristics can be evaluated with the present invention. For example, changes in composite integrity or alterations in crystalline structure over time may be sensed and depicted quantitatively. In practice, a device for non-biological, material-evaluation use will have a menu of applications to select from as part of the initial instrument set up. The use of the present invention is demonstrated in the following examples.

### EXAMPLE 1

In this example, 110 axillary lymph nodes dissected from breast-cancer patients were analyzed. Of these nodes, 17 were cancerous and 93 non-cancerous. Analysis results for the 110 axillary lymph nodes are presented in Table 1 with scan volume being the global variable for this example.

TABLE 1

| Variables | ROC AUC |
|---|---|
| Spectral variables | 0.706 +/ 0.070 |
| Spectral variables + | 0.725 +/− 0.070 |
| scan volume | |
| Spectral variables + | 0.877 +/− 0.048 |
| scan volume + | |
| envelope statistics | |

In this example, using only QUS-variable values derived from spectrum analysis resulted in an ROC AUC value of 0.706+/0.070. Adding the global variable of scan volume to the analysis improved the performance to an ROC AUC value of 0.725+/−0.070. Finally, adding envelope statistics to the analysis further improved the performance to an ROC AUC value of 0.877+/−0.048. A comparison of ROC AUC values for spectral variables to the corresponding AUC values for spectral variables with envelope statistics and a global variable value shows a significant improvement in identification of cancerous nodes.

### EXAMPLE 2

In another example, 289 dissected lymph nodes of mixed primary-cancer types were analyzed. Of these nodes, 43 were histologically proven to be positive and 246 were proven to be negative for cancer. The mixed nodes included 110 breast-cancer nodes with 17 proven to be positive and 93 proven to be negative plus 179 colorectal-cancer nodes with 26 proven to be positive and 153 proven to be negative for cancer. Table 2a shows results obtained with a non-linear classifier—a support-vector machine (SVM)—while Table 2b shows results for a linear classifier—linear discriminant analysis (LDA). The variables investigated include a QUS variable derived from spectrum analysis, a QUS variable derived from envelope statistics and two distinct global variables: primary cancer type and scan volume (proportional to lymph-node volume). The non-linear analysis (Table 2a) shows that adding global variables significantly improves classifier performance over the spectral variable alone. Similarly, adding the envelope-statistics further improves the performance. Table 2b shows the same improvement trend when global and envelope-statistics variables are added. Finally, comparing Tables 2a and 2b demonstrates how non-linear classification methods can perform better than linear methods.

TABLE 2a
SVM-based Classification

| Variables | ROC AUC |
|---|---|
| Spectral variables | 0.66 +/− 0.04 |
| Spectral variables + | 0.71 +/− 0.04 |
| primary cancer type | |
| Spectral variables + | 0.79 +/− 0.03 |
| primary cancer type + | |
| scan volume | |
| Spectral variables + | 0.87 +/− 0.02 |
| primary cancer type + | |
| scan volume + | |
| envelope statistics variables | |

TABLE 2b
Linear-discriminant-based Classification

| Variables | ROC AUC |
|---|---|
| Spectral variables | 0.70 +/− 0.04 |
| Spectral variables + | 0.68 +/− 0.04 |
| primary cancer type | |
| Spectral variables + | 0.78 +/− 0.03 |
| primary cancer type + | |
| scan volume | |
| Spectral variables + | 0.78 +/− 0.03 |
| primary cancer type + | |
| scan volume + | |
| envelope statistics variables | |

### EXAMPLE 3

### Non-Biological Material

To demonstrate how the invention can be used in a non-tissue-typing approach, radio-frequency ultrasound data were collected from two phantoms (PA and PB) using a single-element transducer operating at 10 MHz. The data were processed to yield two QUS-variable estimates associated with the backscattered spectrum (i.e., spectral intercept and spectral slope) and two additional QUS variables associated with the Nakagami envelope statistics model. In both cases, correction was made for the different attenuation of the two phantoms. The optional global variable was not used for this example. Table 3 summarizes the ability of these variables to distinguish between PA and PB.

TABLE 3

| Variables | ROC AUC |
|---|---|
| QUS Spectrum | 0.75 |
| QUS Spectrum + Envelope | 0.95 |

The results demonstrate that the envelope variables significantly increase classification performance over spectral variables alone.

Although the present invention has been described in conjunction with specific embodiments, those of ordinary skill in the art will appreciate the modifications and variations that can be made without departing from the scope and the spirit of the present invention. Such modifications and variations are envisioned to be within the scope of the appended claims.

1. A method of classifying non-biological material comprising:
acquiring ultrasound, pulse-echo, backscattered RF signals from the non-biological material being evaluated;
computing spectral-variable values from said RF echo signals;
computing estimates of scatterer properties (such as effective scatterer size, or acoustic concentration) from said spectral-variable values;
computing additional variable values of the envelope statistics of said RF signals in terms of defined statistical models;
inputting said spectral-variable values, said scatterer-property-estimate values, and said envelope-statistics variable values into a classifier;
assigning a classifier-score value to each of a plurality of variable values, wherein each said classifier-score value for each variable-value combination indicates the relative likelihood of a given material property.

2. The method of claim 1 in which an optional global variable is inputted into said classifier.

3. The method of claim 1 wherein said statistical variables are computed using a Nakagami distribution model.

4. The method of claim 1 wherein said statistical variables are computed using a homodyned-K distribution model.

5. A method of classifying biological tissue comprising:
acquiring ultrasound, pulse-echo, backscattered, RF echo signals from the biological material being evaluated;
computing spectral-variable values from said RF echo signals;
computing additional variable values of the envelope statistics of said RF signals in terms of defined statistical models;
inputting said spectral-variable values, and said envelope-statistics-variable values into a classifier;
assigning a classifier-score value to each of a plurality of classifier variables, wherein each said property value for each assigned variable indicates the likelihood of a given material property.

6. The method of claim 1 in which an optional global variable is inputted into said classifier.

7. The method of claim 5 wherein said statistical variables are extracted using a Nakagami distribution model.

8. The method of claim 7 wherein said statistical variables are extracted using a homodyned-K distribution model.

9. A method of classifying material comprising:
acquiring ultrasound, pulse-echo, backscattered, RF echo signals from the material being evaluated;
computing spectral-variable values from said RF echo signals;
computing estimates of scatterer properties (such as effective scatterer size, or acoustic concentration) from said spectral-variable values;
inputting global-variable values, said spectral-variable values, and said scatterer-property-estimate values into a classifier;
assigning a classifier-score value to each of a plurality classifier variables wherein each said property value for each assigned variable indicates the relative likelihood of a given material property.

10. The method of claim 9 wherein said statistical variables are extracted using a Nakagami distribution model.

11. The method of claim 9 wherein said statistical variables are extracted using a homodyned-K distribution model.

12. The method of claim 9 in which an optional global variable is inputted into said classifier.
